(19) United States
(12) Patent Application Publication
(10) Pub. No.: US 2007/0100323 A1
(43) Pub. Date: May 3, 2007

(54) Facilitation of endothelialization by in situ surface modification

(76) Inventors: Florian Niklas Ludwig, Mountain View, CA (US); Syed Faiyaz Ahmed Hossainy, Fremont, CA (US); Fozan EI-Nounou, Santa Clara, CA (US); A. Adam Sharkawy, San Jose, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/264,514

(22) Filed: Oct. 31, 2005

(51) Int. Cl. A61M 31/00; A01N 63/00; A61F 13/00; A01N 65/00

(52) U.S. Cl. 604/509; 424/422; 424/93.7

(57) ABSTRACT

A method including delivering to a treatment site within a lumen of a blood vessel (1) a cellular component including at least one of endothelial cells and endothelial progenitor cells and/or (2) a conjugate having a first site having affinity to or capable of conjugating with the blood vessel and a second site having affinity to a cellular component or capable of conjugating with a cellular component. A method including coating a treatment site within a lumen of a blood vessel with a polymeric biomaterial including (1) molecular moieties with affinity to cells or a treatment agent and (2) a treatment agent. A composition including a cellular component including endothelial cells or endothelial progenitor cells and modified to increase the potential for retention at a treatment site. A composition including a polymeric biomaterial including (1) molecular moieties with affinity to cells or a treatment agent or (2) a treatment agent.

### BACKGROUND

1. Field of the Invention

Inhibiting intravascular thrombosis, vascular smooth muscle cell proliferation or restenosis.

2. Background

Balloon angioplasty is utilized as an alternative to bypass surgery for treatment early in the development of stenosis or occlusion of blood vessels due to the abnormal build-up of plaque on the endothelial wall of a blood vessel. Angioplasty typically involves guiding a catheter that is usually fitted with a balloon through an artery to the region of stenosis or occlusion, followed by brief inflation of the balloon to push the obstructing intravascular material or plaque against the endothelial wall of the vessel, thereby compressing and/or breaking apart the plaque and reestablishing blood flow. In some cases, particularly where a blood vessel may be perceived to be weakened, a stent may be deployed.

Balloon angioplasty and stent deployment may result in injury to a wall of a blood vessel and its endothelial lining. For example, undesirable results such as denudation (removal) of the endothelial cell layer in the region of the angioplasty, dissection of part of the inner vessel wall from the remainder of the vessel wall with the accompanying occlusion of the vessel, or rupture of the tunica intima layer of the vessel. A functioning endothelial reduces or mitigates thrombogenicity, inflammatory response, and neointimal proliferation.

### SUMMARY

According to one embodiment of the invention that may be used to reduce the risk of intravascular thrombosis formation, and/or it may be used to inhibit vascular smooth muscle cell proliferation or restenosis following, for example, vascular intervention or injury, or in denuded or incompletely endothelialized areas of vasculature. One way the method achieves this is by accelerating recovery of endothelial coverage by delivering to a treatment site within a lumen of a blood vessel, a cellular component including either or both of endothelial cells and endothelial progenitor cells. The cellular component may be modified (e.g., genetically modified) to increase expression of molecules capable of attaching to a wall of a blood vessel. Alternatively, it may be encapsulated in a lipid or biodegradable polymer membrane capable of lodging in openings or fissures at the injury site, or modified at its surface to attach to a wall of a blood vessel. Still further, the cellular component may be modified to express or release a treatment agent such as a growth factor or a cytokine. In still another embodiment, the cellular component may be modified, for example, at its surface, to include a molecule or molecular moiety that either or in conjunction with a compatible molecule is capable of attaching the cellular component to a wall of a blood vessel.

In addition to accelerating endothelial coverage area, the functionality of the endothelial cells may also be facilitated. As endothelial density up to confluence and inter-cellular communication influences endothelial function, improved or accelerated recovery of endothelial function may also be achieved by increasing the rate of recovering endothelial coverage. While a confluent coverage of a functionally competent endothelium is a desired outcome, the method may be deemed successful in any instance where vascular healing mediated by facilitation of re-endothelialization is improved.

According to another embodiment, the method includes delivering to an injury site within a lumen or a blood vessel, a treatment agent having a first site capable of adhering with a wall of a blood vessel and a second site capable of bonding or conjugating with a cellular component. By utilizing a treatment agent having a second site having an affinity for endothelial progenitor cells, the conjugates may attract circulating cells (e.g., endothelial progenitor cells) from the blood stream, either those cells naturally present or cells introduced (infused locally or systemically) in or after a procedure.

According to another embodiment, a method is also described. The method includes coating an injury site within a lumen of a blood vessel with a polymeric biomaterial. The biomaterial may contain molecular moieties with affinity to cells and/or a treatment agent. For example, a coating may present molecular moieties at its luminal surface with affinity to the surface of circulating progenitor cells or cells locally infused after coating the blood vessel wall. The coating may be loaded with a treatment agent, such as a cytokine and/or growth factor to stimulate migration of neighboring cells to the injury site or impede proliferation of target cells (e.g., smooth muscle cells). Additionally the coating may also be loaded with cytokines or growth factors such as, for example, vascular endothelial growth factor (VEGF), fibroblast growth factor (FGF), platelet-derived growth factor (PDGF) to stimulate recovery of endothelial coverage and/or function. Alternatively or in addition, the composition from the culture expanded media for endothelial coverage may be added to the coating. This will include an array of cytokines, growth factors to elicit a synergistic effect. In another embodiment, a treatment agent may be embedded in the coating through the use of carriers such as polymer particles, liposomes and polymer vesicles. Alternatively, a coating may incorporate a treatment agent by providing attachment site for the treatment agent. The treatment agent, in such case, may disassociate from the attachment site with a certain dissolution rate or may be chemically conjugated to the biomaterial through a degradable bond, releasing the treatment agent upon degradation.

One property of a polymeric biomaterial coating of a blood vessel is that it may insulate a vessel wall from platelets deposition and monocyte/neutrophil adhesion. In order to increase a thrombo-resistant property of the biomaterial coating, drugs such heparin may be incorporated into the coating.

According to still another embodiment, a composition is described that may include an amount of a cellular component suitable for delivery to a blood vessel. The cellular component may include endothelial cells or progenitor cells (e.g., endothelial progenitor cells) that have been modified to increase the potential for retention at a treatment site within the blood vessel. Modifications include but are not limited to, genetic or molecular modifications to increase the retention of molecules at a treatment site (e.g., affinity for a blood vessel wall), encapsulation in lipid or polymer membranes or shells with affinity to the vessel wall, expressing or releasing agents that stimulate migration of neighboring cells to a treatment site or impede proliferation of target cells. In another embodiment, a composition is disclosed that is suitable for being introduced at a treatment site and has a property capable of capturing or recruiting circulating cells, such as circulating progenitor cells.

In a further embodiment, a composition including a polymeric biomaterial is described. The polymeric biomaterial is suitable for delivery into a blood vessel possibly to form an in situ coating on a wall of the blood vessel. The biomaterial may include moieties with affinity to circulating cells and/or treatment agents such as cytokines, growth factors or drugs. The embodiment includes but is not limited to the following coating configurations: a) Hydrogel materials containing bioactive agent, that are packaged in nanoparticle or nanovesicular form; b) a blend of hydrophilic and hydrophobic polymers such as polyethylene glycol (PEG) and d,l-polylactic acid (d,l-PLA) such that the blend contains and allows the transport of bioactive agents into the tissue and prevents platelet activation by virtue of, for example, a PEG rich surface. The blend ratio may be optimized based on four parameters: transport of bioactive, surface hydrophilicity without any polyion, interfacial adhesion to the tissue, and the kinetics of dissolution or disintegration of the coating.

### BRIEF DESCRIPTION OF THE DRAWINGS

The features, aspects, and advantages of the invention will become more thoroughly apparent from the following detailed description, appended claims, and accompanying drawings in which:

FIG. 1 shows a schematic side and sectional view of a blood vessel.

FIG. 2 shows a cross-sectional side view of a distal portion of a catheter assembly in a blood vessel during an angioplasty procedure.

FIG. 3 shows the blood vessel of FIG. 2 following the removal of the catheter assembly.

FIG. 4 schematically illustrates a cellular component of a treatment agent modified to express a moiety to binding sites on a blood vessel.

FIG. 5 schematically illustrates a vessel wall modified to recruit circulating cells.

FIG. 6 schematically illustrates a bioconjugation between two conjugates on a cellular component and a blood vessel wall, respectively.

FIG. 7 shows a representation of a cross-linking event involving multifunctional molecular moieties.

FIG. 8 shows the blood vessel of FIG. 3 and a first embodiment of a catheter assembly to deliver a treatment agent introduced into the blood vessel.

FIG. 9 shows the blood vessel of FIG. 3 and a second embodiment of a catheter assembly to deliver a treatment agent introduced into the blood vessel.

FIG. 10 shows the blood vessel of FIG. 3 and a third embodiment of a catheter assembly to deliver a treatment agent introduced into the blood vessel.

FIG. 11 shows the blood vessel of FIG. 3 and a fourth embodiment of a catheter assembly to deliver a treatment agent introduced into the blood vessel.

FIG. 12 shows the blood vessel of FIG. 3 and a fifth embodiment of a catheter assembly to deliver a treatment agent introduced into the blood vessel.

FIG. 13 shows a sixth embodiment of a catheter assembly to deliver a treatment agent introduced into the blood vessel.

FIG. 14 shows a seventh embodiment of a catheter assembly to deliver a treatment agent introduced into the blood vessel.

FIG. 15 shows an eighth embodiment of a catheter assembly to deliver a treatment agent introduced into the blood vessel.

FIG. 16 shows a ninth embodiment of a catheter assembly to deliver a treatment agent introduced into the blood vessel.

FIG. 17 shows a tenth embodiment of a catheter assembly to deliver a treatment agent introduced into the blood vessel.

FIG. 18 shows the blood vessel of FIG. 3 and a eleventh embodiment of a catheter assembly to deliver a treatment agent introduced into the blood vessel.

FIG. 19 shows the blood vessel of FIG. 3 and a twelfth embodiment of a catheter assembly to deliver a treatment agent introduced into the blood vessel.

FIG. 20 shows the blood vessel of FIG. 3 and a thirteenth embodiment of a catheter assembly to deliver a treatment agent introduced into the blood vessel.

### DETAILED DESCRIPTION

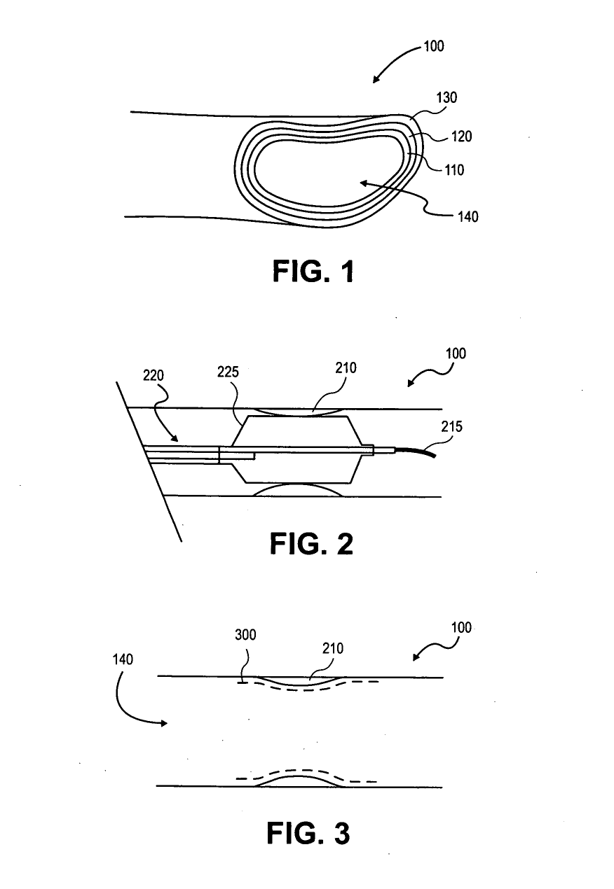

Referring to FIG. 1, a non-diseased artery is illustrated as a representative blood vessel. Blood vessel 100 includes an arterial wall having a number of layers. Inner most layer 110 is generally referred as to the intimal layer. that includes the endothelium, the subendothelial layer, and the internal elastic lamina. Medial layer 120 is concentrically outward from inner most layer 110 and bounded by external elastic lamina. There is no external elastic lamina in a vein. Medial layer 120 (in either an artery or vein) primarily consists of smooth muscle fibers and collagen. Adventitial layer 130 is concentrically outward from medial layer 120. The arterial wall (including inner most layer 110, medial layer 120 and adventitial layer 130) defines lumen 140 of blood vessel 100.

Stenosis or occlusion of a blood vessel such as blood vessel 100 occurs by the build-up of plaque on inner most layer 110. The stenosis or occlusion can result in decreased blood flow through lumen 140. One technique to address this is angioplasty. FIG. 2 shows a portion of artery of blood vessel 100 including stenosis or occlusion referenced herein as injury site or treatment site 210. To minimize or remove the stenosis or occlusion, catheter assembly 220 including balloon 225, may be advanced over guidewire 215 to the injury site (treatment site). Balloon 225 may be briefly inflated one or more times to dilate the vessel and/or minimize the size of the stenosis or occlusion. FIG. 2 shows balloon 225 in an expanded state contacting and exerting pressure on treatment site 210. The dilating of a vessel or minimizing of a stenosis or occlusion may restore blood flow in blood vessel 100 to levels approaching those prior to the formation of the stenosis or occlusion.

FIG. 3 shows blood vessel 100 following an angioplasty procedure. Representatively, the stenosis or occlusion at treatment site 210 is minimized and portions of plaque contributing to the stenosis or occlusion may have been removed. FIG. 3 also shows the optional deployment of a structural supporting device or stent 300 over stenosis or occlusion of treatment site 210.

Balloon angioplasty and stent deployment may result in injury to blood vessel 100 and its endothelial lining, resulting in a potential formation of thrombus or neointimal proliferation. A functioning endothelial reduces or mitigates thrombogenecity, inflammatory response, neointimal proliferation. Therefore, it is desirable to accelerate re-endothelialization.

One technique for accelerating re-endothelialization at an injury site within a blood vessel is to infuse a cellular component that promotes the growth of endothelial cells or a restoration of an endothelial layer. In one embodiment, the technique includes introducing endothelial cells or progenitor cells (e.g., endothelial progenitor cells) as, for example, a fluid local to the target site to repopulate an injured vessel wall and/or stent (or other blood contacting implant) surface. The cell source may be autologous, meaning perhaps that it was previously harvested from the blood stream of the patient undergoing a procedure. In such case, the endothelial cells or endothelial progenitor cells (EPC) harvested from the blood stream may be concentrated and then fused locally to an injury site. Alternatively, the cell source may be an exogenous cell line, such as an allogenic (human) cell line not from the patient.

Treatment Agents

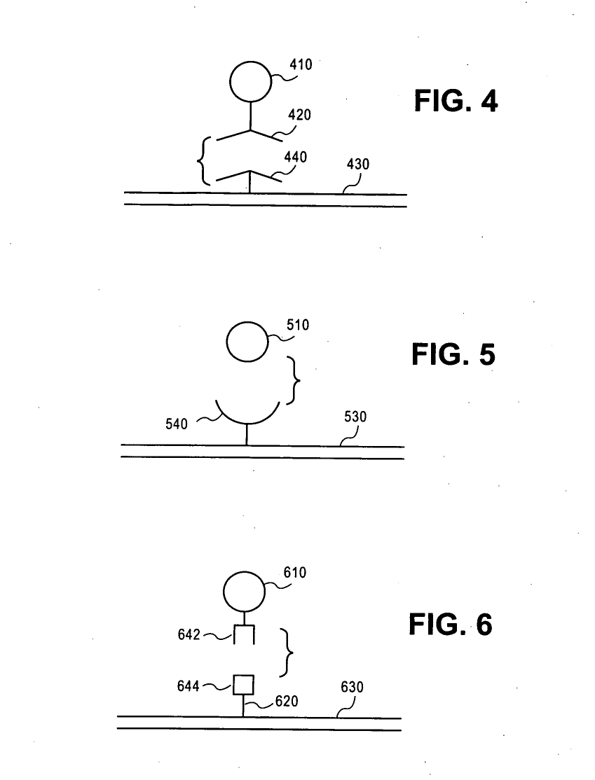

In one embodiment, a treatment agent may include suitable cellular components such as but not limited to endothelial cells or progenitor cells (e.g., endothelial progenitor cells) or bone marrow derived stem cells or other stem cells that may have a property or function that modifies (e.g., improves) a blood vessel wall following an injury to the vessel wall such as may occur in the context of a treatment of a stenosis or occlusion, or in the context of naturally occurring incompletely endothelialized vasculature. In another embodiment, these cells may be pre-conditioned to increase the expression of attachment molecules, where such attachment molecules have, for example, affinity to a subendothelium. For example, cells may be genetically modified to express integrins or other moieties that have an affinity to binding sites of the subendothelium such as proteins present therein, e.g. laminin, collagen or fibrin, or the arginine-glycine-aspartic acid (RGD) sequence found in these proteins. FIG. 4 schematically illustrates a cellular component of a treatment agent modified to express a moiety to binding sites on a blood vessel. FIG. 4 shows cell 410 expressing moiety 420. Moiety 420 has an affinity for a protein or amino acid sequence 440 (e.g., an RGD sequence) of vessel wall 430.

In another embodiment, the cells may be packaged or encapsulated. in a liposome or stealth liposome or other outer shell such as, for example, lipid or polymer membranes, or polymer shells, or other lipid-philic shells. This embodiment recognizes that an atheromatous plaque tends to have a number of micro-cracks in its surface. These micro-cracks can act as secondary reservoirs for cell component treatment agents. Accordingly, an embodiment where a cell component treatment agent is packaged or encapsulated in liposomes or other outer shell such as lipid or polymer membranes, or polymer shells, or other lipid-philic shells, allows the treatment agent to be lodged into the micro-cracks of the atheromatous plaque. In order to accommodate endothelial progenitor cells, these cell-loaded capsules are, for example, of a size of 10 to 20 micrometers (μm).

In another embodiment, cells such as endothelial cells or endothelial progenitor cells may be genetically modified to express and/or release a therapeutic in situ. For example, a cell may be modified to express and/or release a cytokine capable of stimulating migration toward a particular site or a growth factor (e.g., VEGF) having a tendency to make cells proliferate. Such cytokines or growth factors may help to induce migration and/or proliferation of neighboring endothelial cells to an injured (e.g., denuded) vessel wall surface or help to recruit circulating endothelial progenitor cells to the target site. A modification of cells to express and/or release a therapeutic may be done in conjunction with a modification to the cell to increase the expression of attachment molecules or the packaging of cells in liposomes or other membrane capsules with a surface having affinity to the subendothelium of a vessel wall.

In another embodiment, cells may be modified, for example, at their surface, by bi- or multifunctional linker molecules where at least one functionality of the linker molecule has affinity to the cell surface of molecular components thereof, and at least one other functionality has affinity to the surface of the target site, e.g., the subendothelium. For example, a molecule having two linked antibodies, where one antibody has affinity to a receptor on a cell surface and the other antibody has affinity to proteins of the subendothelium may be used to modify the cells. Alternatively, antibody fragments, affibodies (a library of proteins with recognition capabilities similar to antibodies), peptides or other molecules with the desired affinity may be used. For example, an anti-CD34 antibody linked to an affibody with affinity to an RGD sequence via a short organic spacer may be used to modify the surface of endothelial progenitor cells. When modifying progenitor cells in this fashion, the CD34 antibody will adhere to CD34 receptors at the cell surface and the cell surface will present molecules (affibodies) with affinity to proteins of the subendothelium (e.g., RGD sequences present in proteins of the subendothelium). In another example, anti-laminin-anti-CD34 may be used to modify the cell surface. Alternatively, suitable linker molecules, having an affinity to the subendothelium, may be chemically conjugated to a cell surface, such as to amine groups using reactive esters, epoxides, aldehydes; to sulfhydryl groups using maleimides, vinyl sulfones; to carboxyl groups using dimethylaminopropylcarbodiimide (EDC) chemistry. A molecular moiety having affinity for a target area such as a lumen surface may be separated from the attachment site on the cell surface by a spacer. Representative spacers include hydrophilic polymers such as polyethylene glycol (PEG) of molecular weight from about, but are not intended to be limited to, 500 to 40,000, preferably from about 2,000 to 10,000, and more preferably, from about 2,000 to 5,000.

In a situation where a stent is deployed at a target site, it may be desirable to deliver a treatment agent including a cellular component to the stent surface. Delivery to a stent surface may be enhanced by using a stent the surface of which is coated with cell adhesion molecules, such as laminin, fibronectin, or an adhesion peptide such as RGD peptide. The treatment agent may be modified in a manner specified above to enhance adhesion or improve the therapeutic activity of the treatment agent. Alternatively, a stent surface may be modified to include morphological features to provide a means of cell adhesion enhancement.

Modification of Blood Vessel

In the above embodiments, compositions and devices for introducing compositions are described, where the compositions may be introduced as a treatment agent into a blood vessel or to a stent in a blood vessel to, for example, promote endothelial cell migration or proliferation at a target site. In another embodiment, a treatment agent may be introduced that has a capability to recruit cells to a target site such as a luminal surface of the blood vessel. These treatment agents may include properties capable of recruiting circulating endothelial progenitor cells, either those naturally present or those cells infused to a target site. One technique to recruit cells to a target site is to modify a vessel wall to retain such cells. FIG. 5 schematically illustrates a vessel wall modified to recruit circulating cells. FIG. 5 shows vessel wall 530 having a surface modified to contain molecule or moiety 540 that has a property that makes it capable of attracting cell 510.

Bi- or multifunctional linkers or molecules have at least one functionality having affinity to a surface of the lumen surface of the target site, such as an affinity for proteins of the subendothelium (e.g., laminin, fibronectin, collagen, tissue factor) and at least one other functionality having affinity to the surface of endothelial cells or progenitor cells, or molecular components present at the respective cell surface. One example of a molecule having affinity to a surface of circulating endothelial cells is a CD34 antibody. An example of a bi-functional molecule is an anti-CD34 -anti-RGD molecule. When infused into a target area, the anti-RGD moiety of this molecule can attach to proteins (e.g., laminin, fibrin) present at a denuded lumen surface, thereby presenting the anti-CD34 moiety to the vessel lumen. When circulating endothelial progenitor cells come in contact with the modified lumen surface, the CD34 receptor of the cell can attach to the CD34 antibody, thereby effectively retaining the cell at the modified target surface. An additional example would be the fab-fragment of an anti-CD133 antibody conjugated to an anti-laminin antibody. Alternatively, a vessel wall may be coated with an anti-laminin-anti-CD34 or an anti-laminin-anti-CD133 molecule by inducing either of these molecules local to a target site.

Molecules or molecular moieties possessing affinity to a surface of endothelial progenitor cells may be chemically conjugated through a luminal surface of a target site. The molecule or molecular moiety may be conjugated to the lumen surface via a spacer molecule, such as a hydrophilic polymer (e.g., PEG) to enhance accessibility. In one embodiment, the molecular moiety may possess more than one molecular moiety with affinity to a cell surface wherein a spacer may be, for example, branched.

Alternatively, attachment molecules may be chemically conjugated to the luminal surface of a blood vessel, through, for example: (i) amine groups using reactive esters, epoxides, aldehydes, or isocyanates (NCO); (ii) sulfhydryl groups using maleimides, vinyl sulfones; (iii) carboxyl groups using dimethylaminopropylcarbodiimide chemistry; (iv) hydroxyl groups using isocyanates (NCO) or epoxides. In this embodiment, one of the functionalities of the bi-functional molecule consists of a chemically reactive group while the other functionality of the molecule has affinity, for example, to the surface of endothelial or endothelial progenitor cells. Alternatively, photo-reactive chemistry may be used for conjugation. An example of a photo-reactive conjugation involves activating a photo-reactive moiety of a molecule by a catheter-based light or ultraviolet (UV) radiation after infusion of the molecular into a target lumen.

One example of modifying a vessel wall with an agent capable of recruiting cells at a target site is modifying a lumen surface of a blood vessel with antibodies to receptors present on endothelial progenitor cells (e.g., CD34, CD133, KDR). This may be done by conjugating a vinyl sulfone(VS)-PEG-antibody molecule to sulfhydryl groups present at a lumen surface. VS-PEG-antibody molecule may be locally infused or circulated in a lumen volume isolated by proximal and distal occlusion balloon (e.g., see FIG. 9) to modify the lumen surface. A VS-PEG-antibody molecular construct may be made in the following way. A cystein residue may be inserted at a C terminus of an antibody (e.g., CD34, CD133), or an antibody fragment by genetic engineering. Genetic code of antibodies may be obtained from clonol selection through phage display. The genetic code of a mono-clonol antibody may be modified to include a cystein residue at the C terminus and expressed in a bacterial or mammalian expression system as described in, for example, Harma, et al., Clinical Chemistry (2000), 46:1755-61. These engineered antibodies may be incubated with a molar excess of VS-PEG-VS to yield a sulfhydryl reactive, via-PEG-antibody.

In another example, a maleimide-anti-CD133 molecule may be used to conjugate the CD133 antibody to sulfhydryl groups present in the vessel lumen surface. Alternatively, an NHS-PEG-biotin may be conjugated to the subendothelium at a target site and avidin may be subsequently infused into the target area. In a final step, VEGF-biotin may be bound to the vessel wall by infusing it into the avidin-modified target area. VEGF has affinity to the KDR receptor found on the surface of endothelial progenitor cells. Alternatively, biotinylated anti-CD34 may be used in the last step.

In yet another example, cyclic RGD (cRGD) molecules may be infused to the treatment site, where the cRGD non-specifically adheres to the subendothelial matrix, thereby providing attachment sites for endothelial cells or endothelial progenitor cells.

Modification of Blood Vessel Wall and Cellular Component

In the above embodiments, compositions may, for example, be introduced into a blood vessel as treatment agents to promote endothelial cell migration or proliferation at a target site. For example, treatment agents including cellular components that have been modified to increase affinity for a luminal wall of a blood vessel or treatment agent that has affinity to a cell surface (e.g., endothelial progenitor cells) may be introduced into a blood vessel. In a further embodiment, a treatment agent (first treatment agent) may be introduced into a blood vessel that has affinity for a cell surface without affinity for a luminal surface of the blood vessel at approximately the same time, after or prior to the introduction of a treatment agent (second treatment agent) with no affinity for a cell surface, but with affinity to the luminal surface of the blood vessel. In such case, the first treatment agent, in addition to being modified to have an affinity for a wall surface may be modified to present a conjugate and the second treatment agent may have a corresponding conjugate so that the first treatment agent and the second treatment agent may be conjugated through a bioconjugate of a conjugate on the first treatment agent and the conjugate on the second treatment agent. FIG. 6 schematically illustrates the bioconjugation. Representatively, first treatment agent 610 of an endothelial progenitor cell may be modified to present conjugate 642 such as avidin chemically conjugated to treatment agent 610 such as through amine groups, sulfhydryl groups, or carboxyl groups. Second treatment agent 620 may be modified to have affinity for a luminal surface of blood vessel wall 630, such as an affinity to binding sites of the sub-endothelium such as RGD sequences found in laminin, collagen or fibrin. Second treatment agent 620 also has conjugate 644 chemically connected thereto that has an affinity for conjugate 642 of first treatment agent 610. A suitable conjugate in this example is, for example, biotin.

Blood Vessel Wall Modification

In another embodiment, a luminal surface of a vessel wall may be modified at a treatment site by forming a coating on the luminal surface of, for example, a hydrogel. In one embodiment, this modification or coating may be formed in situ. Suitable hydrogels include, but are not limited to, cross-linked PEG hydrogels or hydrogels formed from biopolymers. One example of a hydrogel that may be formed in situ (e.g., within a lumen of a blood vessel) is the combination of tri- or more functional PEG-amine with bi- or more functional PEG-reactive ester at a slightly basic pH (e.g., on the order of 7.6 to 9.0). Another example of a suitable hydrogel is a hydrophilic polymer such as PEG or a biopolymer such as chitosan mixed with a photoreactive crosslinker. A suitable photoreactive crosslinker is, for example, is a bi- or multifunctional acrylate where site specific photo-irradiation will locally activate the crosslinker to form a localized hydrogel.

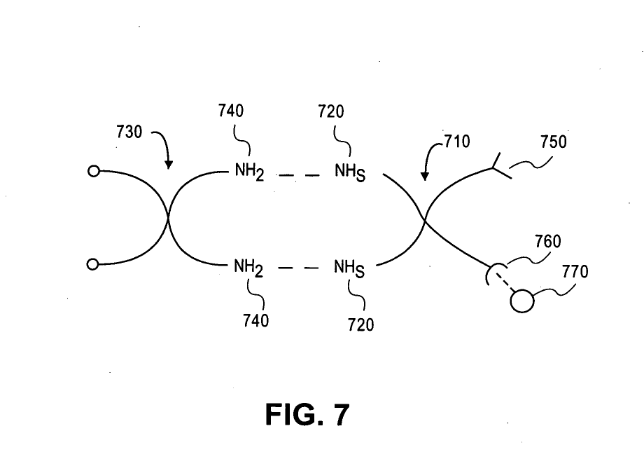

FIG. 7 shows a representation of a cross-linking event involving a multifunctional PEG-amine with a multifunctional PEG-reactive ester. FIG. 7 shows multifunctional PEG-ester moiety 710 having reactive ester groups 720 at ends of two chains. Those reactive esters are available for bonding to reactive amines of multifunctional PEG-amine moiety 730. FIG. 7 shows esters (NHS ester groups) of multifunctional PEG-ester moiety 710 aligned with amine groups of multifunctional PEG-amine moiety 730. In addition to forming a hydrogel in situ, the hydrogel may present molecular moieties at a luminal surface of the blood vessel having affinity for constituents of the vessel wall (e.g., peptides or fractions of subendothelial proteins such as RGD sequences). FIG. 7 shows multifunctional PEG-ester moiety 710 having molecular moiety 750 with an affinity for a luminal surface of a blood vessel. Alternatively, or in addition, a hydrogel may present molecular moieties at a luminal surface of the gel with an affinity for circulating cellular components, such as circulating progenitor cells. FIG. 7 shows multifunctional PEG-ester moiety 710 having molecular moiety 760 (e.g., CD34, CD133, KDR) with affinity for circulating progenitor cells 770.

In addition to having molecular moieties to promote the adhesion of the hydrogel to a vessel wall or a hydrogel with an affinity for circulating progenitor cells, a hydrogel may be loaded with a therapeutic agent, such as a cytokine and/or growth factor to stimulate migration of neighboring endothelial cells to the target area, or a therapeutic agent to impede proliferation of target cells, e.g., smooth muscle cells. Drug carriers such as polymer particles, liposomes, or polymer vesicles may be embedded in the hydrogel. Alternatively, the hydrogel may incorporate the therapeutic by providing attachment sites for the therapeutic, for example, where the therapeutic disassociates from these attachment sites with a certain disassociation rate. Or, the therapeutic may be chemically conjugate to the polymers of the hydrogel where the chemical bond is degradable, releasing the therapeutic upon degradation. FIG. 7 may be illustrative of this concept with molecular moiety 760, for example, being substituted with an attachment site for a therapeutic agent.

In one embodiment, a hydrogel formed in situ on a vessel wall, such as a denuded vessel wall may insulate the vessel wall from platelet deposition and monocyte/neutrophil adhesion. In order to increase a thrombo-resistant property of the hydrogel, an inhibitor such as heparin may be incorporated into the coating. A hydrogel coating may also contain a cocktail of acellular components of culture expansion in order to induce controlled healing.

Cellular components may be delivered as described above after a vessel wall has been modified, such as by a hydrogel coating. Cellular components may include mature endothelial cells or progenitor cells (e.g., endothelial progenitor cells). The affinity of a hydrogel for a particular cell may be modified using techniques described above (e.g., presenting moieties in the hydrogel that have an affinity for a particular cell).

In addition to combining surface modification of a wall coating such as a hydrogel with affinity for cellular components and cell delivery, a vessel coating modification and cell surface modification may be used as a complement. For example, a surface of a wall coating (e.g., a hydrogel) may be modified to present a conjugate and a separate cellular component may be modified at its surface or genetically modified to express surface receptors to present a conjugate or molecular moiety having an affinity for the conjugate presented by the wall coating. The conjugation of a conjugate on the wall coating and a conjugate or other molecular moiety on the surface of the cell will form a bioconjugate. An alternative to a chemical conjugation or binding, these conjugates or conjugate and molecular moieties may be in the form of magnetically responsive materials.

One example of the above description is modifying the surface of endothelial progenitor cells by incubation with NHS-PEG-biotin and subsequent incubation in avidin. At the same time, a surface of a wall coating is modified by NHS-PEG-biotin alone. Thus, avidin is attached to the cell surface, or biotin is presented at the lumen surface. When the avidin-modified cells are infused into a target area and the cell surface bound avidin is brought into contact with the lumen-bound biotin, the avidin will bind the biogen and thereby retain the cells at the lumen surface.

Devices

In the above embodiments, treatment agents including a cellular component and modified treatment agents are described that may be used to modify (e.g., improve) a target site such as luminal surface of a blood vessel. Also described are treatment agents having a capability to recruit cells to a target site or to modify a target site such as by coating a luminal surface of a blood vessel. The following paragraphs describe representative devices that may be used to introduce the contemplated treatment agents.

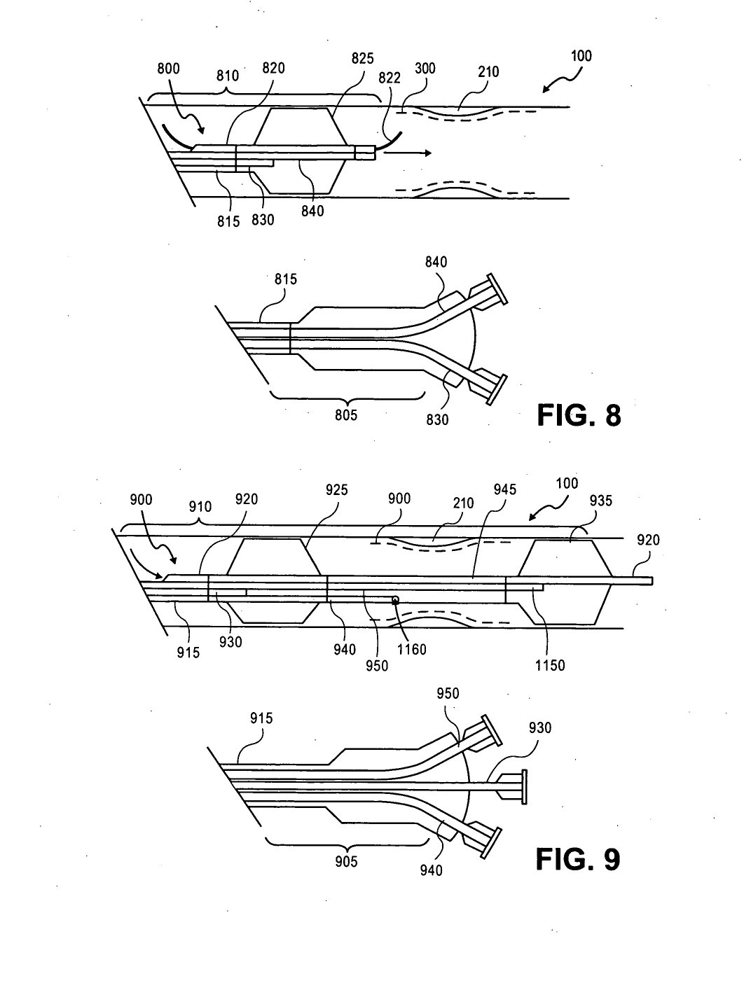

To increase delivery and engraftment efficiency of a treatment agent including, for example, modified or unmodified cells, blood flow may be temporarily reduced or a stopped through balloon occlusion of the target vessel prior to the introduction. FIG. 8 shows blood vessel 100 having catheter assembly 800 disposed therein. Catheter assembly 800 includes proximal portion 805 and distal portion 810. Proximal portion 805 may be external to blood vessel 100 and to the patient. Representatively, catheter assembly 800 may be inserted through a femoral artery and through, for example, a guide catheter and with the aid of a guidewire to a location in the vasculature of a patient. That location may be, for example, a coronary artery. FIG. 8 shows distal portion 810 of catheter assembly 800 positioned proximal or upstream from treatment site 210.

In one embodiment, catheter assembly 800 includes primary cannula 815 having a length that extends from proximal portion 805 (e.g., located external through a patient during a procedure) to connect with a proximal end or skirt of balloon 825. Primary cannula 815 has a lumen therethrough that includes inflation cannula and delivery cannula 840. Each of inflation cannula 830 and delivery cannula 840 extends from proximal portion 805 of catheter assembly 800 to distal portion 810. Inflation cannula 830 has a distal end that terminates within balloon 825. Delivery cannula 840 extends through balloon 825.

Catheter assembly 800 also includes guidewire cannula 820 extending, in this embodiment, through balloon 825 through a distal end of catheter assembly 800. Guidewire cannula 820 has a lumen sized to accommodate guidewire 822. Catheter assembly 800 may be an over the wire (OTW) configuration where guidewire cannula 820 extends from a proximal end (external to a patient during a procedure) to a distal end of catheter assembly 800. Guidewire cannula 820 may also be used for delivery of a treatment agent such as a cellular component or other vessel wall modifying agent when guidewire 822 is removed with catheter assembly 800 in place. In such case, separate delivery cannula (delivery cannula 840) is unnecessary or a delivery cannula may be used to delivery one treatment agent while guidewire cannula 820 is used to delivery another treatment agent.

In another embodiment, catheter assembly 800 is a rapid exchange (RX) type catheter assembly and only a portion of catheter assembly 800 (a distal portion including balloon 825) is advanced over guidewire 822. In an RX type of catheter assembly, typically, the guidewire cannula/lumen extends from the distal end of the catheter to a proximal guidewire port spaced distally from the proximal end of the catheter assembly. The proximal guidewire port is typically spaced a substantial distance from the proximal end of the catheter assembly. FIG. 8 shows an RX type catheter assembly.

In one embodiment, catheter assembly 800 is introduced into blood vessel 100 and balloon 825 is inflated (e.g., with a suitable liquid through inflation cannula 830) to occlude the blood vessel. Following occlusion, a solution (fluid) including a cellular component that promotes the growth of endothelial cells or a restoration of an endothelial layer is introduced through delivery cannula 840. A suitable solution of endothelial cells or progenitor cells is a saline solution with a concentration of endothelial cells or progenitor cells on the order of 10² to 10⁵ per milliliter (ml), more specifically 10³ to 10⁵ per milliliter. By introducing the cellular component in this manner, the endothelial cells or progenitor cells can re-populate the vessel wall at treatment site 210 or stent 300.

In an effort to improve the target area of a cellular component to a treatment site, such as treatment site 210, the injury site may be isolated prior to delivery. FIG. 9 shows an embodiment of a catheter assembly having two balloons where one balloon is located proximal to treatment site 210 and a second balloon is located distal to treatment site 210. FIG. 9 shows catheter assembly 900 disposed within blood vessel 100. Catheter assembly 900 has a tandom balloon configuration including proximal balloon 925 and distal balloon 935 aligned in series at a distal portion of the catheter assembly. Catheter assembly 900 also includes primary cannula 915 having a length that extends from a proximal end of catheter assembly 900 (e.g., located external to a patient during a procedure) to connect with a proximal end or skirt of balloon 925. Primary cannula 915 has a lumen therethrough that includes inflation cannula 930 and inflation cannula 950. Inflation cannula 930 extends from a proximal end of catheter assembly 900 to a point within balloon 925. Inflation cannula 930 has a lumen therethrough allowing balloon 925 to be inflated through inflation cannula 930. In this embodiment, balloon 925 is inflated through an inflation lumen separate from the inflation lumen that inflates balloon 935. Inflation cannula 950 has a lumen therethrough allowing fluid to be introduced in the balloon 935 to inflate the balloon. In this manner, balloon 925 and balloon 935 may be separately inflated. Each of inflation cannula 930 and inflation cannula 950 extends from, in one embodiment, the proximal end of catheter assembly 900 through a point within balloon 925 and balloon 935, respectively.

Catheter assembly 900 also includes guidewire cannula 920 extending, in this embodiment, through each of balloon 925 and balloon 935 through a distal end of catheter assembly. Guidewire cannula 920 has a lumen therethrough sized to accommodate a guidewire. No guidewire is shown within guidewire cannula 920. Catheter assembly 900 may be an over the wire (OTW) configuration or a rapid exchange (RX) type catheter assembly. FIG. 9 illustrates an RX type catheter assembly.

Catheter assembly 900 also includes delivery cannula 940. In this embodiment, delivery cannula extends from a proximal end of catheter assembly 900 through a location between balloon 925 and balloon 935. Secondary cannula 945 extends between balloon 925 and balloon 935. A proximal portion or skirt of balloon 935 connects to a distal end of secondary cannula 945. A distal end or skirt of balloon 925 is connected to a proximal end of secondary cannula 945. Delivery cannula 940 terminates at opening 960 through secondary cannula 945. In this manner, a treatment agent may be introduced between balloon 925 and balloon 935 positioned between treatment site 210.

FIG. 9 shows balloon 925 and balloon 935 each inflated to occlude a lumen of blood vessel 100 and isolate treatment site 210. In one embodiment, each of balloon 925 and balloon 935 are inflated to a point sufficient to occlude blood vessel 100 prior to the introduction of a treatment agent. A treatment agent containing a cellular component of, for example, endothelial cells or progenitor cells (e.g., endothelial progenitor cells) is then introduced.

In the above embodiment, separate balloons having separate inflation lumens are described. It is appreciated, however, that a single inflation lumen may be used to inflate each of balloon 925 and balloon 935. Alternatively, in another embodiment, balloon 935 may be a guidewire balloon configuration such as a PERCUSURG™ catheter assembly where catheter assembly 900 including only balloon 925 is inserted over a guidewire including balloon 935.

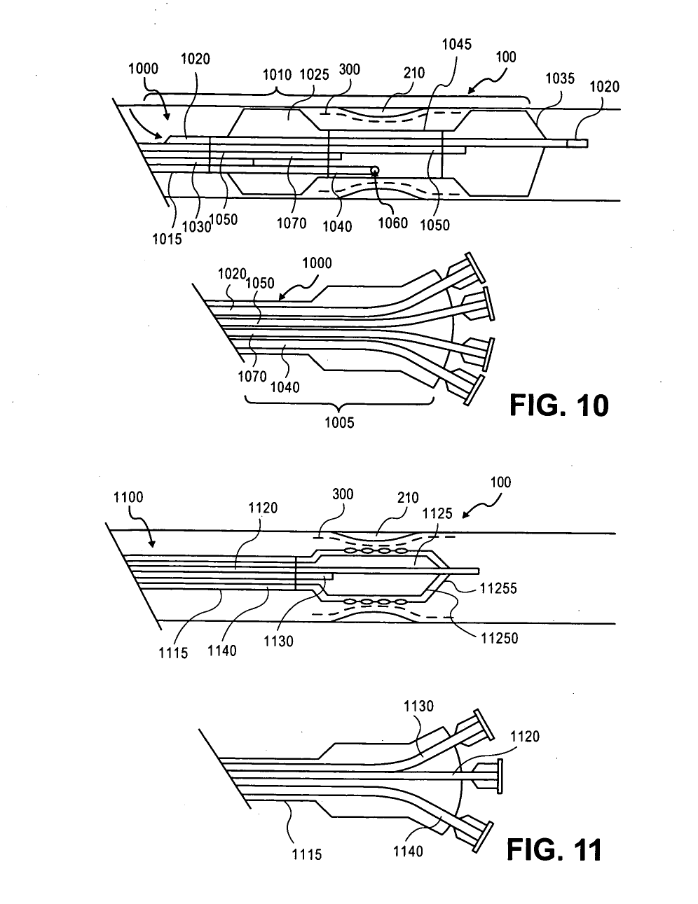

FIG. 10 shows catheter assembly 1000 disposed within a lumen of blood vessel 100. Catheter assembly 1000 has a tandom balloon configuration similar to the configuration described with respect to catheter assembly 900 of FIG. 9. In this case, the secondary cannula between the tandom balloons is also inflatable. FIG. 10 shows catheter assembly 1000 includes primary cannula or tubular member 1015. In one embodiment, primary cannula 1010 extends from a proximal end of the catheter assembly (proximal portion 1005) intended to be external to a patient during a procedure, to a point proximal to a region of interest or treatment site within a patient, in this case, proximal to treatment site 210. Representatively, catheter assembly 1000 may be percutaneously inserted via femoral artery or a radial artery and advanced into a coronary artery.

Primary cannula 1015 is connected in one embodiment to a proximal end (proximal skirt) of balloon 1025. A distal end (distal skirt) of balloon 1025 is connected to secondary cannula 1045. Secondary cannula 1045 has a length dimension, in one embodiment, suitable to extend from a distal end of a balloon located proximal to a treatment site beyond a treatment site. In this embodiment, secondary cannula 1045 has a property such that it may be inflated to a greater than outside diameter than its outside diameter when it is introduced (in other words, secondary cannula 1045 is made of an expandable material). A distal end of secondary cannula 1045 is connected to a proximal end (proximal skirt of balloon 1035). In one embodiment, each of balloon 1025, balloon 1035, and secondary cannula 1045 are inflatable. Thus, in one embodiment, each of balloon 1025, balloon 1035, and secondary cannula 1045 are inflated with a separate inflation cannula. FIG. 10 shows catheter assembly having inflation cannula 1030 extending from a proximal end of catheter assembly 1000 to a point within balloon 1025; inflation cannula 1050 extending from a proximal end of catheter assembly 1000 to a point within balloon 1035; and inflation cannula 1070 extending from a proximal end of catheter assembly 1000 to a point within secondary cannula 1045. In another embodiment, the catheter assembly may have a balloon configured in a dog-bone arrangement such that inflation of the balloon through a single inflation lumen inflates each of what are described in the figures as separated balloon 1025, balloon 1035 and secondary cannula 1045.

By using an expandable structure such as secondary cannula 1045 adjacent a treatment site, the expandable structure may be expanded to a point such that a treatment agent may be dispensed very near or at the treatment site. FIG. 10 shows catheter assembly 1000 including delivery cannula 1040 extending from a proximal end of catheter assembly 1000 through primary cannula 1015, through balloon 1025 and into secondary cannula 1045. Delivery cannula 1040 terminates at dispensing port 1060 within secondary cannula 1045. As viewed, secondary cannula 1045 is expandable to an outside diameter less than an expanded outside diameter of balloon 1025 or balloon 1035 (e.g., secondary cannula 1045 has an inflated diameter less than an inner diameter of blood vessel 100 at a treatment site).

FIG. 11 shows another embodiment of a catheter assembly. Catheter assembly 1100, in this embodiment, includes a porous balloon through a treatment agent, such as endothelial cells or progenitor cells (e.g., endothelial progenitor cells) may be introduced. FIG. 11 shows catheter assembly 1100 disposed within blood vessel 100. Catheter assembly 1100 has a porous balloon configuration positioned at treatment site 210. Catheter assembly 1100 includes primary cannula 1115 having a length that extends from a proximal end of catheter assembly 1100 (e.g., located external to a patient during a procedure) to connect with a proximal end or skirt of balloon 1125. Primary cannula 1115 has a lumen therethrough that includes inflation cannula 1130. Inflation cannula 1130 extends from a proximal end of catheter assembly 1100 to a point within balloon 1125. Inflation cannula 1130 has a lumen therethrough allowing balloon 1125 to be inflated through inflation cannula 1130.

Catheter assembly 1100 also includes guidewire cannula 1120 extending, in this embodiment, through balloon 1125. Guidewire cannula 1120 has a lumen therethrough sized to accommodate a guidewire. No guidewire is shown within guidewire cannula 1120. Catheter assembly 1100 may be an over-the-wire (OTW) configuration or rapid exchange (RX) type catheter assembly. FIG. 11 illustrates an OTW type catheter assembly.

Catheter assembly 1100 also includes delivery cannula 1140. In this embodiment, delivery cannula 1140 extends from a proximal end of catheter assembly 1100 to proximal end or skirt of balloon 1125. Balloon 1125 is a double layer balloon. Balloon 1125 includes inner layer 11250 that is a non-porous material, such as PEBAX, Nylon or PET. Balloon 1125 also includes outer layer 11255. Outer layer 11255 is a porous material, such as extended polytetrafluoroethylene (ePTFE). In one embodiment, delivery cannula 1140 is connected to between inner layer 11250 and outer layer 11255 so that a treatment agent can be introduced between the layers and permeate through pores in balloon 1125 into a lumen of blood vessel 100.

As illustrated in FIG. 11, in one embodiment, catheter assembly is inserted into blood vessel 100 so that balloon 1125 is aligned with treatment site 210. Following alignment of balloon 1125 of catheter assembly 1100, balloon 1125 may be inflated by introducing an inflation medium (e.g., liquid through inflation cannula 1130). In one embodiment, balloon 1125 is only partially inflated or has an inflated diameter less than an inner diameter of blood vessel 100 at treatment site 210. In this manner, balloon 1125 does not contact or only minimally contacts the blood vessel wall. A suitable expanded diameter of balloon 1125 is on the order of 2.0 to 5.0 mm for coronary vessels. It is appreciated that the expanded diameter may be different for peripheral vasculature. Following the expansion of balloon 1125, a treatment agent, such as a cellular component of endothelial cells or progenitor cells (e.g., endothelial progenitor cells) is introduced into delivery cannula 1140. The treatment agent flows through delivery cannula 1140 into a volume between inner layer 11250 and outer layer 11255 of balloon 1125. At a relatively low pressure (e.g., on the order of two to four atmospheres (atm)), the treatment agent then permeates through the porous of outer layer 11255 into blood vessel 100.

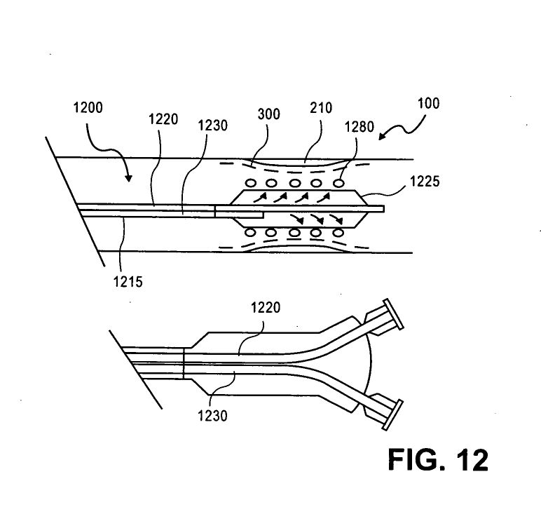

FIG. 12 shows another embodiment of a catheter assembly suitable for introducing a treatment agent into a blood vessel. FIG. 12 shows catheter assembly 1200 disposed within blood vessel 100. Catheter assembly 1200 includes primary cannula 1215 having a length that extends from a proximal end of catheter assembly 1200 (e.g., located external to a patient during a procedure) to connect with a proximal and/or skirt of balloon 1225. Balloon 1225, in this embodiment, is located at a position aligned with treatment site 210 in blood vessel 100.

Disposed within primary cannula 1215 is guidewire cannula 1220 and inflation cannula 1230. Guidewire cannula 1220 extends from a proximal end of catheter assembly 1200 through balloon 1225. A distal end or skirt of balloon 1225 is connected to a distal portion of guidewire cannula 1220.

Inflation cannula 1230 extends from a proximal end of catheter assembly 1200 to a point within balloon 1225. In one embodiment, balloon 1225 is made of a porous material such as ePTFE. A suitable pore size for an ePTFE balloon material is on the order of one micron (μm) to 60 μms. The porosity of ePTFE material can be controlled to accommodate a treatment agent flow rate or particle size by changing a microstructure of an ePTFE tape used to form a balloon, for example, by wrapping around a mandrel. Alternatively, pore size may be controlled by controlling the compaction process of the balloon, or by creating pores (e.g., micropores) using a laser.

ePTFE as a balloon material is a relatively soft material and tends to be more flexible and conformable with tortuous coronary vessels than conventional balloons. ePTFE also does not need to be folded which will lower its profile and allow for smooth deliverability to distal lesions and the ability to provide therapy to targeted or regional sites post angioplasty and/or stent deployment.

A size of balloon 1225 can also vary. A suitable balloon diameter is, for example, in the range of two to five millimeters (mm). A balloon length may be on the order of eight to 60 mm. A suitable balloon profile range is, for example, approximately 0.030 inches to 0.040 inches.

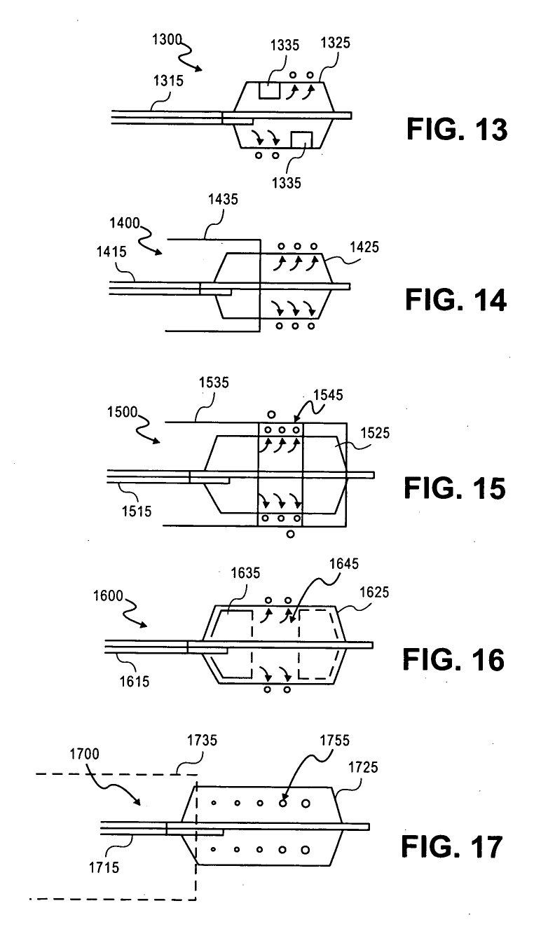

In one embodiment, a porous balloon may be masked in certain areas along its working length to enable more targeted delivery of a treatment agent. FIG. 13 shows an embodiment of porous balloon masked in certain areas. Catheter assembly 1300 includes balloon 1325 connected to primary cannula 1315. Balloon 1325 is a porous material such as ePTFE with masks 1335 of a nonporous material (e.g., Nylon) positioned along a working length of balloon 1325.

In another embodiment, a sheath may be advanced over a porous balloon (or the balloon withdrawn into a sheath) to allow tailoring of a treatment agent distribution. FIG. 14 shows catheter assembly 1400 including balloon 1425 connected to primary cannula 1415. Sheath 1435 is located over a portion of balloon 1425 (a proximal portion of the working length).

In another embodiment, a sheath may have a window for targeting delivery of the treatment agent through a porous balloon. FIG. 15 shows catheter assembly 1500 including balloon 1525 connected to primary cannula 1515. Sheath 1535 is extended over a working length of balloon 1525. Sheath 1535 has window 1545 that provides an opening between the sheath and balloon 1525.

In another embodiment, a liner inside a porous balloon may be used to target preferential treatment agent delivery. For example, the liner may have a window through which a treatment agent is delivered, e.g., on one side of a liner for delivery to one side of a vessel wall. This type of configuration may be used to address eccentric lesions. FIG. 16 shows catheter assembly 1600 including balloon 1625 of a porous material connected to primary cannula 1610. Disposed within (e.g., connected to an inner wall of) balloon 1625 is liner 1635 of a non-porous material such as Nylon. FIG. 16 also shows opening or window 1245 between liner portions that allow a material to exit pores in balloon 1625. Alternatively, a liner may have a tailored distribution of pores along the liner. The orientation of the balloon liner may be visualized through radio-opaque markers or through indicators on the external portion of catheter assembly 1600.

In an alternative embodiment, rather than using a porous material like ePTFE for forming a porous balloon (e.g., balloon 1225 in FIG. 12), a conventional balloon material such as PEBAX, Nylon or PET may be used that has tens or hundreds of micropores around its circumference for treatment agent diffusion. A suitable pore size may range, for example, from approximately five to 100 microns. Pores may be created by mechanical means or by laser perforation. Pore distribution along a balloon surface may be inhomogeneous to tailor distribution of treatment agent delivery. For example, FIG. 17 shows catheter assembly 1700 including balloon 1725 connected to primary cannula 1715. Balloon 1725 has a number of openings or pores 1755 extending in a lengthwise direction along the working length of balloon 1725. The pores get gradually larger along its length (proximal to distal). FIG. 17 shows two rows of pores 1755 as an example of a pore distribution. In other examples, pores 1755 may be created only on one side of balloon 1725 to deliver a treatment agent preferentially to one side of a blood vessel (e.g., to address eccentric lesions). The orientation of balloon 1725 in this situation may be visualized through radio-opaque markers, or through indicators on an external portion of catheter assembly 1700. Balloon 1725 may also be retractable into optional sheath 1735 to tailor a length of treatment agent delivery. In an alternative embodiment, sheath 1735 may have an opening on one side to preferentially deliver a treatment agent to one side of the vessel.

According to any of the embodiments described with reference to FIGS. 12-17 and the accompanying text, a treatment agent such as a cellular component including endothelial cells or progenitor cells (e.g., endothelial progenitor cells) may be introduced through the inflation cannula (e.g., inflation cannula 1230) to expand the balloon (e.g., balloon 1225). In the example of a balloon of a porous material, such as balloon 1225, the treatment agent will expand balloon 1225 and at relatively low pressure (e.g., 2-4 atm) diffuse through pores in the porous balloon material to treatment site 210 within a lumen of blood vessel 100. FIG. 12 shows treatment agent 1280 diffusing through balloon 1225 into a lumen of blood vessel 100. Since balloon 1225 is positioned at treatment site 210, treatment agent 1280 is diffused at or adjacent (e.g., proximal or distal) to treatment site 210.

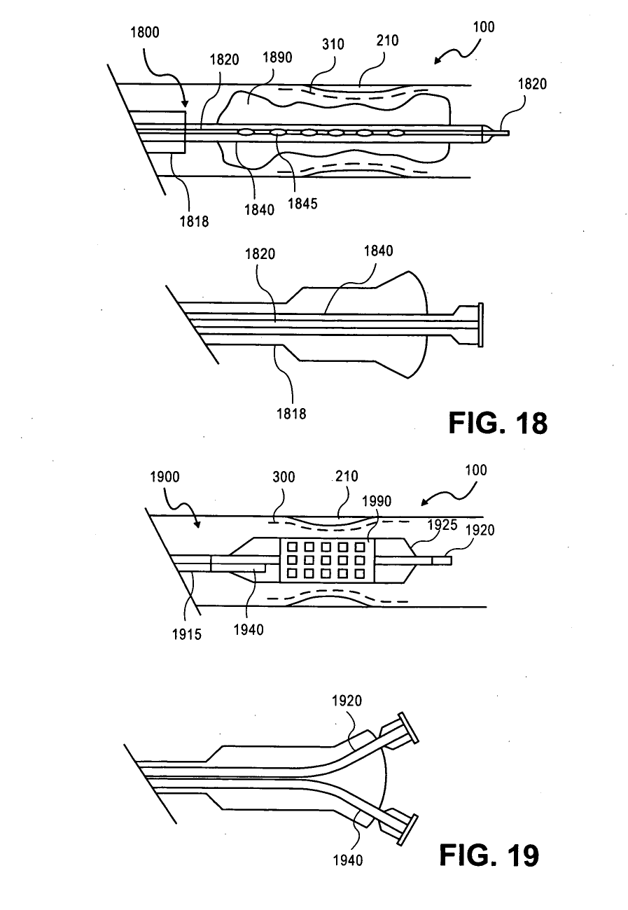

FIG. 18 shows another embodiment of a catheter assembly suitable for introducing a treatment agent at a treatment site. FIG. 18 shows catheter assembly 1800 disposed within blood vessel 100. In this embodiment, catheter assembly 1800 utilizes an absorbent possibly porous device such as a sponge or a brush, connected to a catheter to dispense a treatment agent.

In one embodiment, catheter assembly 1800 includes guidewire cannula 1820 extending from a proximal end of catheter assembly 1800 (e.g., external to a patient during a procedure) to a point in blood vessel 100 beyond treatment site 210. Overlying guidewire cannula 1820 is primary cannula 1840. In one embodiment, primary cannula 1840 has a lumen therethrough of a diameter sufficient to accommodate guidewire cannula 1820 and to allow a treatment agent to be introduced through primary cannula 1840 from a proximal end to a treatment site. In one embodiment, catheter assembly 1800 includes a brush or sponge material connected at a distal portion of primary cannula 1840. A sponge is representatively shown. Sponge 1890 has an exterior diameter that, when connected to an exterior surface of primary cannula 1840 will fit within a lumen of blood vessel 100. Catheter assembly 1800 also includes retractable sheath 1818 overlying primary cannula 1840. During insertion of catheter assembly 1800 into a blood vessel to a treatment site, sponge 1890 may be disposed within sheath 1818. Once catheter assembly 1800 at a distal portion disposed at a treatment site, sheath 1818 may be retracted to expose sponge 1890. FIG. 18 shows sheath 1818 retracted, such as by pulling the sheet in a proximal direction.

In one embodiment, prior to insertion of catheter assembly 1800, sponge 1890 may be loaded with a treatment agent. Representatively, sponge 1890 may be loaded with a cellular component including endothelial cells and progenitor cells (e.g., endothelial progenitor cells).

In one embodiment, catheter assembly 1800 may provide for additional introduction of a treatment agent through primary cannula 1840. FIG. 18 shows primary cannula 1840 having a number of dispensing ports 1845 disposed in series along a distal portion of primary cannula 1840 coinciding with a location of sponge 1890. In this manner, once sponge 1890 is placed at treatment site 210 within blood vessel 100, additional treatment agent may be introduced through primary cannula 1840 if desired.

FIG. 19 shows another embodiment of a catheter assembly suitable for introducing a treatment agent into a blood vessel. FIG. 19 shows catheter assembly 1900 disposed within blood vessel 100. Catheter assembly 1900 includes primary cannula 1915 having a length that extends from a proximal end of catheter assembly 1900 (e.g., located external to a patient during a procedure) to connect with a proximal end or skirt of balloon 1925. Balloon 1925, in this embodiment, is located at a position aligned with treatment site 210 in blood vessel 100.

In one embodiment, catheter assembly 1900 has a configuration similar to a dilation catheter, including guidewire cannula 1920 and inflation cannula 1940 disposed within primary cannula 1915. Guidewire cannula 1920 extends through balloon 1925 and balloon 1925 is connected to a distal end or skirt of guidewire cannula 1920. Inflation cannula 1940 extends to a point within balloon 1925.

In one embodiment, catheter assembly 1900 includes sleeve 1990 around a medial working length of balloon 1925. Balloon 1925, including a medial working length of balloon 1925, may be made of a non-porous material (e.g., a non-porous polymer). In one embodiment, sleeve 1990 is a porous material that may contain a treatment agent such as a cellular component as described above. A representative material for sleeve 1990 is a silastic material. Sleeve 1990 may be loaded with or soaked (e.g., saturated) in a treatment agent before inserting catheter assembly 1900 into a blood vessel. Representatively, the pores of the porous sleeve may be filled with agent beforehand. The pores can also expand upon balloon inflation to deliver payload.

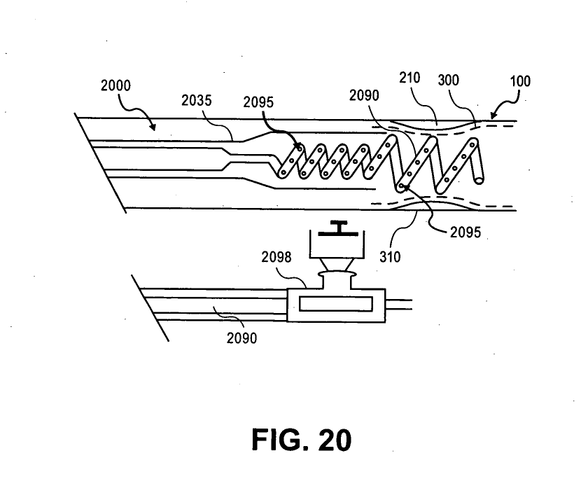

FIG. 20 shows another embodiment of a catheter assembly suitable for dispensing a treatment agent into a blood vessel. The catheter assembly of FIG. 20 relies on a flexible polymeric or metal hollow coil with microporous perfusion holes to deliver a treatment agent into a blood vessel. FIG. 20 shows catheter assembly 2000 including coil 2090 disposed from a proximal end of the catheter assembly (e.g., intended to be external to a patient during a procedure) to a point within a blood vessel, such as treatment site 210 of blood vessel 100. In one embodiment, coil 2090 is formed from a material that has a hollow cross-section, such as a hypo-tube or extrusion. In the embodiment shown, only a distal portion of coil 2090 is coiled, with the remaining portion being linear. A representative length of a distal portion of coil 2090 is on the order of one to 15 centimeters (cm). In addition, coil may be tapered from proximal to distal having (e.g., a reduced diameter at a distal end) to accommodate narrowing of blood vessels towards distal portion. Alternatively, coil may be in linear configuration in sheath (during delivery before deployment and during catheter retraction after deployment). This may be achieved by using a shape memory material such as Nitinol.

At a distal portion of coil 2090 (e.g., the coiled portion), a number (e.g., hundreds) of perfusion holes or micropores 2095 are formed to release a treatment agent therethrough. A suitable hole or micropore diameter is on the order of five to 100 microns formed, for example, around a circumference of a distal portion of coil 2090 using a laser. A proximal end of coil 2090 is connected to delivery hub 2098. A treatment agent, such as a treatment agent including a cellular component, can be injected through delivery hub 2098 and exit through holes or micropores 2095.

Catheter assembly 2000 includes sheath 2035. Sheath 2035 may be used to deliver coil 2090 to a treatment site and then retracted to expose at least a portion of the distal portion of coil 2090 including holes or micropores 2095. For delivery to a treatment site, a distal end of coil 2090 is tightly wound in either a clockwise or counterclockwise configuration. For delivery of a treatment agent, a distal portion of coil 2090 may be unwound, either by inflation through pressurization or through re-expansion into a previously memorized shape (e.g., where coil is a shape-memory material such as a nickel-titanium alloy). After a treatment agent has been introduced through pores 2095, a distal portion of coil 2090 may be withdrawn, either by deflation or by withdrawal into sheath 2035.

To minimize potential trauma to a vessel wall by shearing of the coil and against the vessel wall, a distal end of coil 2090 may be rounded or have a small sphere. Alternatively, two coils of opposite helisity may be joined at their distal end but not at overlaps in between. In another embodiment, the delivery system may consist of joined “Vs” which are rolled into a cylindrical configuration around an axis orthogonal to a plane of the Vs. Tightly wound in this configuration, a catheter assembly may be delivered to a treatment site where it is unwound to deliver a treatment agent through pores incorporated into the system.

In any of the embodiments of utilizing a coil to deliver a treatment agent, a pore distribution along a distal portion of the coil may be non-uniform to deliver the treatment agent preferentially to specific sites within a treatment area (e.g., to one side of a blood vessel). Techniques for forming coil 2090 include extruding tubing where certain treatment agents such as drugs can be mixed with extrusion resin and then herically slitting the tubing to form a coil. Alternatively, coil 2090 may be made from a hollow ripen.

A flexibility and profile of coil 2090 allows for regional treatment agent delivery in one embodiment up to approximately 15 centimeters long in a coronary vessel. An outer diameter of a hollow coil can range from 0.005 inches to 0.010 inches, and a wall thickness may range from 0.0005 inches to 0.003 inches. Treatment agent distribution may be controlled by pitch length of coil 2090.

The above delivery devices and systems are representation of devices that may be used to deliver a treatment agent including, but not limited to, a modified or unmodified cellular component or treatment agents to modify a luminal surface of a blood vessel. For example, treatment agents suitable to form an in situ layer for wall modification described above with reference to FIG. 7 may be introduced at a treatment site with a variety of delivery devices. These devices include delivery through pores of a porous balloon, see FIGS. 11-12 and the accompanying text, or through a saturated sponge mounted on a distal end of a delivery system, see, for example, FIG. 13. In addition, the vessel may be balloon occluded proximal and distal to the target site as shown in FIG. 9 and FIG. 10 (e.g., a dog-bone shape balloon). Additional treatment agents that might be added subsequently to an in situ formed layer may be deposited through the same deposition devices that are used to introduce the hydrogel coating or through a second devices.

In the preceding detailed description, reference is made to specific embodiments thereof. It will, however, be evident that various modifications and changes may be made thereto without departing from the broader spirit and scope of the following claims. The specification and drawings are, accordingly, to be regarded, in an illustrative rather than a restrictive sense.

What is claimed is:

1. A method comprising:
delivering to an treatment site within a lumen of a blood vessel by a percutaneous transluminal route at least one of (1) a cellular component comprising at least one of endothelial cells and endothelial progenitor cells and (2) a conjugate having a first site capable of conjugating with a wall of the blood vessel and a second site capable of conjugating with a cellular component.

2. The method of claim 1, wherein the cellular component that is delivered is modified to increase expression of molecules capable of attaching to a wall of the blood vessel.

3. The method of claim 1, wherein the cellular component that is delivered is packaged in a lipid-philic shell capable of lodging in openings at the injury site.

4. The method of claim 1, wherein the cellular component that is delivered is modified to express or release a treatment agent.

5. The method of claim 4, wherein the treatment agent comprises one of a growth factor and a cytokine.

6. The method of claim 1, wherein the cellular component that is delivered is modified to include a molecule that either alone or in conjunction with a compatible molecule is capable of conjugating the cellular component to a wall of the blood vessel.

7. The method of claim 1, wherein the second site of the conjugate has an affinity for a surface of endothelial progenitor cells.

8. The method of claim 1, wherein prior to delivering the one of the cellular component and the conjugate, the method comprises:
occluding the blood vessel at a point upstream of the injury site.

9. The method of claim 1, wherein prior to delivering the one of the cellular component and the conjugate, the method comprising:
occluding the blood vessel at a point upstream of the injury site and a point downstream of the injury site.

10. The method of claim 1, wherein prior to delivering the one of the cellular component and the conjugate, the method comprises occluding the blood vessel at the injury site with a porous occlusion device, and delivering comprises delivering the treatment agent through the porous occlusion device.

11. The method of claim 1, wherein prior to delivering the at least one of the cellular component and the conjugate, the method comprises inserting an occlusion device into the blood vessel that at least partially occludes the blood vessel, and delivering comprises delivering the agent through the occlusion device.

12. The method of claim 11, wherein the occlusion device comprises a balloon assembly comprising a porous portion and delivering comprises delivering the one of the cellular component and the conjugate through the porous portion.

13. The method of claim 12, wherein a portion of the porous portion is impeded.

14. The method of claim 12, wherein the balloon assembly comprises a non-porous medial working length and a sleeve of a porous material loaded with the at least one of the cellular component and the conjugate.

15. The method of claim 1, wherein prior to delivering the at least one of the cellular component and the conjugate, the method comprises inserting an absorbent device into the blood vessel that is loaded with the at least one of the cellular component and the conjugate.

16. The method of claim 1, wherein prior to delivering the at least one of the cellular component and the conjugate, the method comprises inserting a delivery device into the blood vessel, the deliver device comprising an elongate portion defining an axis and a distal portion coiled at least in part about the axis, the distal portion comprising a plurality of perfusion holes through which the at least one of the cellular component and the conjugate may pass.

17. The method of claim 1, wherein the second site is capable of conjugating with a cellular component comprises an affinity to at least one of CD34 and CD133.

18. A method comprising:
coating a treatment site within a lumen of a blood vessel with a polymeric biomaterial comprising at least one of (1) molecular moieties with affinity to cells or a treatment agent and (2) a treatment agent.

19. The method of claim 18, wherein the treatment agent comprises a property to stimulate migration of cells to the injury site.

20. The method of claim 18, wherein the treatment agent comprises a property to impede migration of cells to the injury site.

21. The method of claim 18, wherein the molecular moieties have an affinity to cells circulating in a blood stream.

22. The method of claim 21, wherein the cells comprise endothelial progenitor cells.

23. The method of claim 18, wherein the treatment agent is formed in a carrier embedded in the biomaterial.

24. A composition comprising:
an amount of a cellular component suitable for delivery into a blood vessel, the cellular component comprising at least one of endothelial cells and endothelial progenitor cells and is modified to increase the potential for retention at a treatment site within the blood vessel.

25. The composition of claim 24, wherein the cellular component is genetically modified to increase expression of molecules capable of attaching to a wall of the blood vessel.

26. The composition of claim 24, wherein the cellular component is modified by packaging in a lipid-philic shell capable of lodging in fissures in a vessel wall at the treatment site.

27. The composition of claim 24, wherein the cellular component is modified to express or release a treatment agent.

28. The composition of claim 27, wherein the treatment agent comprises one of a growth factor and a cytokine.

29. The composition of claim 24, wherein the cellular component is modified to include a molecule that either alone or in conjunction with a compatible molecule is capable of conjugating the cellular component to a wall of the blood vessel.

30. A composition comprising:
a polymeric biomaterial suitable for delivery into a blood vessel, the biomaterial comprising at least one of (1) molecular moieties with affinity to cells or a treatment agent and (2) a treatment agent.

31. The composition of claim 30, wherein the treatment agent comprises one of a growth factor and a cytokine.

32. The composition of claim 30, wherein the treatment agent comprises a property to impede migration of cells to the treatment site.

33. The composition of claim 30, wherein the treatment agent is formed in a carrier embedded in the biomaterial.

34. The composition of claim 30, wherein the biomaterial comprises attachment sites for the treatment agent and the treatment agent has a property to disassociate from the biomaterial over a time period.

35. The composition of 30, wherein the treatment agent is chemically conjugated to the biomaterial through a degradable conjugate.

36. The composition of claim 30, wherein the molecular moieties have an affinity to cells circulating in a blood stream.

37. The composition of claim 36, wherein the cells are endothelial progenitor cells.

38. The composition of claim 30, wherein the molecular moieties with affinity to cells comprises an affinity to at least one of (1) CD34 and (2) CD133.

39. The composition of claim 30, wherein the treatment agent is formulated in one of a polymeric nanoparticle or microparticle, a lipid or polymer vesicle, and a lipid or polymer micelle.
